(19) United States
(12) Patent Application Publication
(10) Pub. No.: US 2022/0112447 A1
(43) Pub. Date: Apr. 14, 2022

(54) PROCESS OF ROTATIONALLY MIXING A LIQUID COMPOSITION THAT INCLUDES A CONSUMER PRODUCT ADJUNCT INGREDIENT

(71) Applicant: The Procter & Gamble Company, Cincinnati, OH (US)

(72) Inventors: Eric Cauyan AMURAO, Kobe (JP); Boon Ho NG, Beijing (CN); Robert Lee HIGGINSON, Independence, KY (US); Ilse Maria Cyrilla D'HAESELEER, Dendermonde (BE); Joana Andreia LAMEIRAS DOMINGUES, Brussels (BE)

(21) Appl. No.: 17/487,039

(22) Filed: Sep. 28, 2021

(30) Foreign Application Priority Data
Oct. 9, 2020 (WO) CN2020/119944

(51) Int. Cl. C11D 11/00; C11D 3/50; C11D 3/37; B01F 9/00

(52) U.S. Cl. CPC C11D 11/0017; C11D 3/505; B01F 2215/0031; C11D 3/3703; B01F 9/003; C11D 3/3761

(57) ABSTRACT

Processes related to rotationally mixing a liquid composition that includes a consumer product adjunct ingredient, for example by subjecting the composition to multiple rotation cycles that include horizontally rotating the composition in a first direction and in a second direction that is counter to the first direction. Related systems and apparatuses. Processes for making consumer products from compositions thusly mixed.

### FIELD OF THE INVENTION

The present disclosure relates to processes related to rotationally mixing a liquid composition comprising a consumer product adjunct ingredient. The present disclosure also relates to related systems, apparatuses, and processes for making consumer products.

### BACKGROUND OF THE INVENTION

Solid or otherwise insoluble adjunct ingredients for use in consumer products are often manufactured well before they are actually incorporated or formulated into a finished product. These ingredients may be present in an aqueous medium, such as in a slurry or oil-in-water emulsion. These feedstock or premix compositions are often placed in large containers in for storage and/or transport.

However, over time, the compositions can become unhomogenized. Specifically, certain solid or insoluble materials may aggregate or phase-separate; as an example, perfume encapsulates are known to float to the top of the intermediate slurry. If not addressed, adjunct concentration and/or viscosity differences in the composition can lead to inconsistent dosing when making the desired final product, resulting to quality control issues.

Thus, a manufacturer needs to re-homogenize the composition, but it can be challenging to do so in an efficient manner. The containers may be shaken, but doing so can be energetically costly for large containers and may still not result in the desired level of mixing. The container can be opened in order to mix the composition via a recirculation process or an overhead mixing process, but this process runs the risk of contamination by microbes, dust, or even atmospheric gas such as oxygen. Furthermore, recirculation processes require clean-out procedures if multiple containers that contain different ingredients are to be mixed, which can be costly in terms of capital, space, and time.

Thus, there is a need for improved processes and systems to homogenize compositions that contain consumer product adjunct ingredients.

### SUMMARY OF THE INVENTION

The present disclosure relates to processes for rotationally mixing a composition in a sealed container, as well as related systems, apparatuses, and containers.

For example, the present disclosure relates to a process for mixing a composition housed in a sealed container, the process including the steps of: providing a composition in a sealed container, where the composition includes a liquid medium and a consumer product adjunct ingredient that is insoluble in the liquid medium; wherein the composition is present in an amount of (a) about 50 kg to about 5000 kg, and/or (b) an amount of about 50 L to about 5000 L; rotating the sealed container around a vertical axis in a rotating step, where the rotating step includes at least two rotation cycles, where a rotation cycle includes a first step followed by a second step, where the first step includes rotating the container in a first direction around the vertical axis, for from about 5 to about 60 seconds at a maximum speed of from about 20 rpms to about 100 rpms, and where the second step includes rotating the container in a second direction around the vertical axis, for from about 5 to about 60 seconds at a maximum speed of from about 20 rpms to about 100 rpms, where the second direction is counter to the first direction.

The present disclosure also relates to a process of making a consumer product, where the process includes the steps of: mixing a composition by a process according to the present disclosure; and combining the composition with a base composition to make the consumer product.

### BRIEF DESCRIPTION OF THE DRAWINGS

The figures herein are illustrative in nature and are not intended to be limiting.

FIG. 1 shows a system according to the present disclosure that is useful for mixing.

FIG. 2 shows a system according to the present disclosure that is useful for mixing.

FIG. 3 shows a container according to the present disclosure.

FIG. 4 shows two containers, in the shape of cylindrical drums, being disposed on a pallet.

FIG. 5 shows a system according to the present disclosure, where the container is being rotated in a first direction.

FIG. 6 shows a system according to the present disclosure, where the container is being rotated in a second direction.

FIG. 7 show a graph of a rotation cycle according to the present disclosure.

### DETAILED DESCRIPTION OF THE INVENTION

The present disclosure relates to processes and apparatuses that are useful in mixing the contents of certain containers, particularly containers that contain a composition that includes a consumer product adjunct ingredient in a liquid medium. The adjunct ingredient may be relatively insoluble in the liquid medium, and may be present as a solid or a droplet; suitable compositions may include, for example, a perfume encapsulate slurry.

The present disclosure relates to processes that mix such containers (and compositions contained therein) by rotating them in a substantially horizontal plane around a vertical axis. The processes include particular rotation profiles, including ramp-up, ramp-down, and pause times Furthermore, the processes include rotating and counter-rotating the containers (e.g., in a first direction and then a second direction that is opposite or counter to the first direction, such as clockwise and counterclockwise), for example at particular rpms (revolutions per minute). The present disclosure also relates to related apparatuses and/or systems, as well as methods of making consumer products using such systems and processes.

Without wishing to be bound by theory, it is believed that by carefully selecting the rotation profiles, the compositions of the containers, which tend to be rather large, are efficiently mixed in a manner that minimizes or even eliminates contamination opportunities, clean-out requirements, and space/footprint requirements.

The processes, systems, apparatuses, and compositions of the present disclosure are described in more detail below.

As used herein, the articles “a” and “an” when used in a claim, are understood to mean one or more of what is claimed or described. As used herein, the terms “include,” “includes,” and “including” are meant to be non-limiting. The compositions of the present disclosure can comprise, consist essentially of, or consist of, the components of the present disclosure.

The terms “substantially free of” or “substantially free from” may be used herein. This means that the indicated material is at the very minimum not deliberately added to the composition to form part of it, or, preferably, is not present at analytically detectable levels. It is meant to include compositions whereby the indicated material is present only as an impurity in one of the other materials deliberately included. The indicated material may be present, if at all, at a level of less than 1%, or less than 0.1%, or less than 0.01%, or even 0%, by weight of the composition.

As used herein the phrase “fabric care composition” includes compositions and formulations designed for treating fabric. Such compositions include but are not limited to, laundry cleaning compositions and detergents, fabric softening compositions, fabric enhancing compositions, fabric freshening compositions, laundry prewash, laundry pretreat, laundry additives, spray products, dry cleaning agent or composition, laundry rinse additive, wash additive, post-rinse fabric treatment, ironing aid, unit dose formulation, delayed delivery formulation, detergent contained on or in a porous substrate or nonwoven sheet, and other suitable forms that may be apparent to one skilled in the art in view of the teachings herein. Such compositions may be used as a pre-laundering treatment, a post-laundering treatment, or may be added during the rinse or wash cycle of the laundering operation.

Unless otherwise noted, all component or composition levels are in reference to the active portion of that component or composition, and are exclusive of impurities, for example, residual solvents or by-products, which may be present in commercially available sources of such components or compositions.

All temperatures herein are in degrees Celsius (° C.) unless otherwise indicated. Unless otherwise specified, all measurements herein are conducted at 20° C. and under the atmospheric pressure.

In all embodiments of the present disclosure, all percentages are by weight of the total composition, unless specifically stated otherwise. All ratios are weight ratios, unless specifically stated otherwise.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### System for Mixing

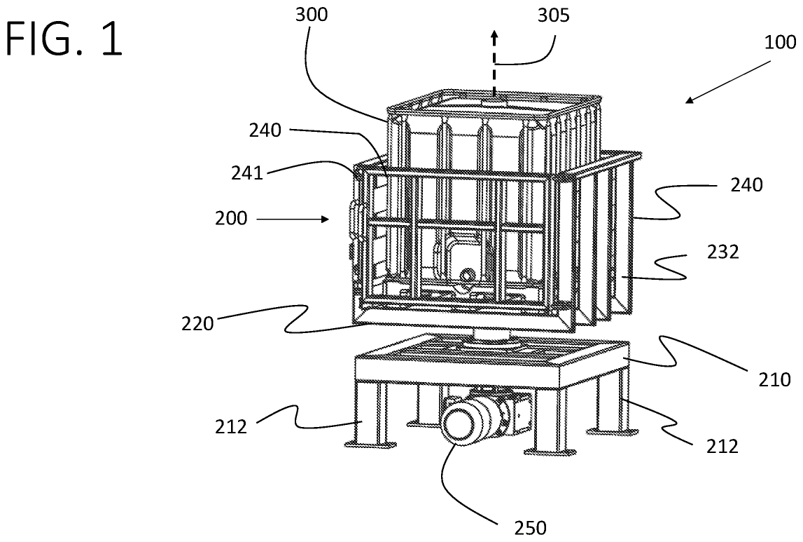

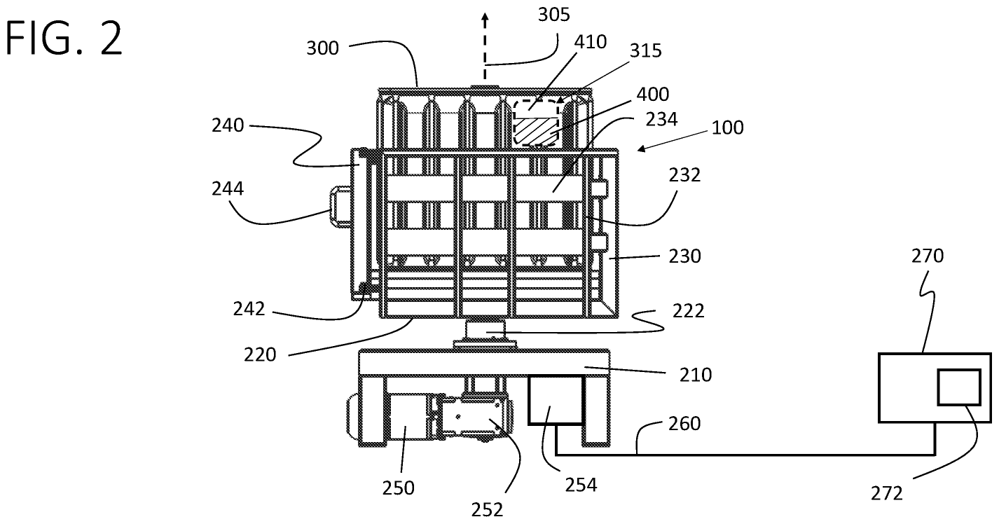

The present disclosure relates to a system 100 useful for mixing. FIGS. 1 and 2 show systems according to the present disclosure. The system 100 includes a mixing apparatus 200 that can rotate a sealed container 300 (or simply “container” as used herein) that houses a composition 400, for example in a manner according to a process of the present disclosure. The mixing apparatus 200, the container 300, and the composition are described in more detail below.

### Mixing Apparatus

The present disclosure relates to a mixing apparatus 200 (or simply “apparatus” as used herein) useful for mixing a composition 400 in a container 300. The apparatus 200 may be used in the processes of the present disclosure.

The mixing apparatus 200 may comprise a frame 210. The frame 210 may comprise one or more legs 212, preferably at least three legs 212, more preferably at least four legs 212, which may be anchored to the ground. The frame should be sturdy enough to support the weight of the filled container, as well as resilient enough to withstand the forces generated from the changes in rotational motion described above. The frame 210 may be a tube steel frame.

The frame 210 may support a deck 220 upon which the container 300 may be placed; the container 300 may be placed upon and rest upon the deck 220. The deck 220 may be capable of rotating in the first direction and the second direction, relative to the frame 210; typically, the frame 210 remains stationary during a rotation cycle. The deck 220 may rotate on a roller bearing 222, preferably a tapered roller bearing.

The deck 220 may include one or more support structures to hold the container 300 in place during a rotation cycle. The one or more support structures may be in the form of a cage 230. The cage 230 may include vertical bars 232. The cage may include horizontal bars 234. The cage 230 may be open at the top as shown in FIGS. 1 and 2, or the cage 230 may be enclosed at the top. A cage 230 may be preferred to solid walls so as to save on materials and/or weight.

The cage 230 may include a gate 240. The gate 240 may be selectively openable. When in an open position, the container 300 may be placed on the deck 220 more easily, for example by a forklift or robot arm. When the container 300 is in place, the gate 240 may be closed to help secure the container 300 in place. The gate 240 may be selectively lockable, for example, by locking means such as a gate latch 241 to further secure the system 100.

The gate 240 may be openable, for example openable about one or more hinges 242. The gate 240 may include a handle 244 to facilitate opening and closing the gate.

A motor 250 powers the rotation of the deck 220 (and by extension, the cage 230, if present). The motor 250 may be sped up and slowed down by a variable frequency drive. A brake may be used to completely stop the motor. A gearbox 252 may be connected to the motor 250 and configured to affect the speed, torque, and direction of the rotation of the deck 220. The apparatus may also include an electrical junction box 254 that houses the electrical connections.

The motor 250 may be in electronic communication, for example via a wired connection 260 to the electrical junction box 254, or even via a wireless connection that uses, for example, wi-fi signals, with a programmable logic computer (PLC) 270. The PLC 270 can be used to program the rotation cycles. The PLC 270 may also have an interface 272 that allows a user to monitor, start, and stop the rotation cycles. The PLC 270 may be located remotely from the motor 250 for safety reasons.

The mixing apparatus 200 is capable of receiving electronic signals that communicate a rotation profile or cycle, for example signals from the PLC 270. The mixing apparatus 200 is capable of carrying out the indicated rotation profile or cycle. Such profiles and cycles are discussed in more detail above.

### Container

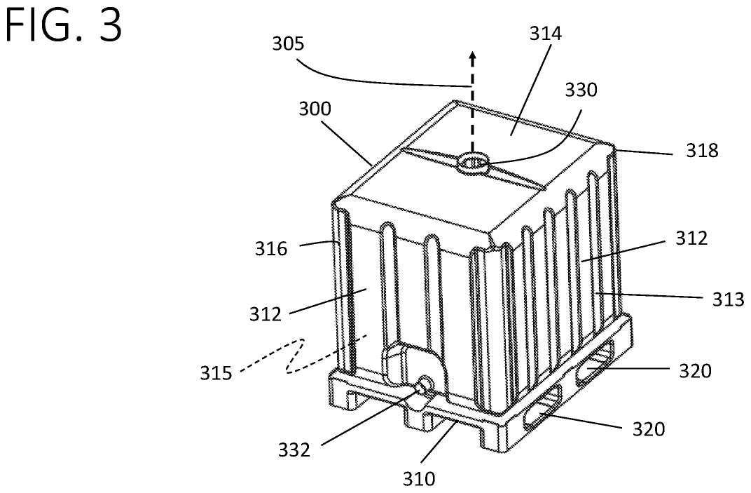

The processes and systems of the present disclosure relate to, among other things, to a container 300. A suitable container 300 is shown in FIG. 3. The container 300 may comprise a composition 400 according to the present disclosure in its internal volume 315.

The container 300 may comprise a base 310. The base may be joined with one or more side walls 312, and the side walls 312 may be joined with a top wall 314, to define an internal volume 315. One or more of the base 310, side walls 312, and/or the top walls 314 may include one or more ribs 313, which can provide structural support to the wall 312 and/or the container 300. The rib 313 may be inwardly projecting (towards the internal volume 315) or outwardly projecting (away from the internal volume 315).

The container 300 may substantially be in the shape of a rectangular prism, or even a cube. The container 300 may be characterized by a footprint that is substantially rectangular. The base 310 may define a footprint that is substantially in the shape of a rectangle. A side wall 312 may meet with the base 310, another side wall 312, or the top wall 314 to form a two-sided corner 316. Two side walls 312 and the base 310, or two side walls 312 and the top wall 314, may meet at a three-sided corner 318. Rectangularly shaped containers may be useful for efficient and safe storage and transport compared to cylindrically shaped containers. For example, they can be packed and stacked with few gaps between them; furthermore, they can be stored upright or on their sides without fear of rolling. Additionally, rectangular containers provide flexibility to the present processes and may not require rigorous quality control with regard to the container's orientation; it may be that the container may be placed in any orientation—e.g., resting on its base 310, on a side wall 312, or even on its top wall 314—and still be rotated and mixed according to the present disclosure.

During mixing, the container 300 may rotate around a vertical axis 305, which may be substantially orthogonal to the top wall 314 and/or the base 310. The vertical axis 305 may pass through the center of the container 300, and/or the center of the top wall 314, and/or the center of the base 310. The base 310 and the top wall 314 may be substantially parallel to each other. It is also understood that when a container 300 is placed on its side, a vertical axis 305 will effectively pass through the side walls 312.

The container 300 may include one or more slots 320, for example integrally formed near the base 310. The slots 320 are openings that may be useful to provide a hold or grip when transporting the container, for example with a forklift. Additionally or alternatively, the container may be coupled with a pallet or other selectively removeable means that is useful for storing and/or transporting the container. If the container 300 is located on a pallet 302, the pallet 302 may comprise slots 303.

The container 300 may include an inlet 330 through which the composition 400 may be provided to the internal volume 315 of the container 300. The inlet 330 may be selectively openable, and in particular, may be sealed so as to protect the composition 400 during transport or storage. The inlet 330 may be sealed airtight once the container 300 is filled. The inlet 330 may be located on a top wall 314 of the container 300.

The container 300 may include an outlet 332 through which the composition 400 may be dispensed from the internal volume 315. The outlet 332 may be selectively openable, and in particular, may be sealed so that the composition 400 does not leak out during transport or storage. The outlet 332 may be sealed airtight until it is time to dispense the composition, for example during the making of a consumer product. The outlet may be located on a side wall 312, preferably near the base 310, of the container. It is understood that although FIG. 3 shows a container 300 with an inlet 330 and an outlet 332, a suitable container may alternatively include a single opening that performs both functions.

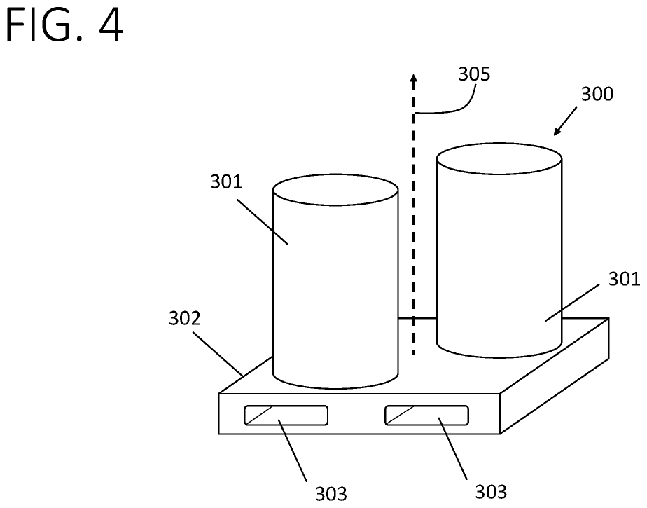

As shown in FIG. 4, a container 300 may be disposed on a pallet 302 or other suitable surface or platform that may be provided to or from the mixing apparatus 200. The pallet 302 may be substantially rectangular in shape. The container 300 may be a drum 301, for example in the general shape of cylinder. FIG. 4 shows two drums 301 being disposed on a pallet 302; however, any suitable number of drums 301 (one, two, three, four . . . ) may be disposed on a pallet 302 so long as they fit and can be adequately secured and rotated according to the presently disclosed process. The pallet 302 that comprises the drums 301 may be rotated around a vertical axis 305, which, depending on the number and placements of the containers 300, may not pass through a container 300, as shown in FIG. 4. The footprint of the pallet 302 may be substantially similar to the footprint of a rectangular prism container, so that both types of containers could be used on the same mixing apparatus 200 with minimal modifications. The pallet 302 may comprise slots 303 sized and configured to receive the forks of a forklift.

The interior surface of the container 300 may be relatively smooth and free of projections that project inwardly towards the vertical axis 305. For example, the container 300 may be free of interior baffles. Although such structures may aid in mixing by creating extra turbulence, they can create extra surface area to which the composition can adhere, making the container 300 harder to empty or clean.

### Compositions

The processes and apparatuses of the present disclosure are useful for mixing liquid compositions 400 that include solid or insoluble ingredients. The compositions 400 are located, provided to, and/or housed in containers 300 according to the present disclosure.

The compositions 400 may comprise consumer product adjunct ingredients. The compositions 400 may be feedstock compositions and/or premix compositions that intended to be combined with other ingredients or base composition to make an intermediate composition or final product composition. The compositions 400 may be prepared at one location and then stored, transported, or a combination thereof prior to making the intermediate or final product composition.

The compositions 400 housed in the containers are generally liquid compositions, meaning that they are generally flowable liquid compositions even though they may contain adjunct ingredients that are insoluble in the liquid medium. When homogenized, the liquid compositions may be characterized by a viscosity of from about 100 to about 10,000 cps, or from about 200 to about 8,000 cps, or from about 300 to about 5000 cps, or from about 500 to about 4000 cps. When homogenized, the liquid composition may have a density of from about 0.8 g/cm³ to about 1.2 g/cm³, or from about 0.9 g/cm³ to about 1.1 g/cm³, or from about 1.0 to about 1.1 g/cm³. Composition viscosity and density are determined according to the methods provided in the Test Methods section below.

The amount of composition 400 housed in the container may be relatively large. For example, the container 300 may comprise from about 50 kg to about 5000 kg, or from about 100 kg to about 4000 kg, or from about 250 kg to about 3000 kg, or from about 500 kg to about 2000 kg, or from about 750 kg to about 1500 kg, or about 800 kg to about 1200 kg, of the composition. The container 300 may comprise from about 50 L to about 5000 L, or from about 100 L to about 4000 L, or from about 250 L to about 3000 L, or from about 500 L to about 2000 L, or from about 750 L to about 1500 L, or about 800 L to about 1200 L, of the composition.

The composition 400 may be present in an amount of at least 80%, or at least about 85%, or at least about 90%, of the internal volume 315 of the container 300. It is believed that relatively full containers are preferred for transport and storage efficiency, as well as to minimize aeration of the compositions contained therein.

That being said, it is typically desirable for there to be some open headspace 410 in the container, which can facilitate improved mixing/homogenization processes, for example by allowing vortexes to form upon rotation. The container 300 may comprise a headspace 410 present at a level of from about 5% to about 20%, preferably from about 10% to about 20%, even more preferably from about 12% to about 18%, by volume, of the internal volume 315 of the container 300. To reduce the chance of microsusceptibility upon storage, it may be preferred that the air in the headspace 410 has been sanitized. FIG. 2 shows a portion of the container 300 being cut away, so that portions of the internal volume 315, the composition 400, and the headspace 410 can be seen.

The composition 400 may comprise a liquid medium. The liquid medium may be present in an amount of at least 50%, or at least 60%, or at least 70%, or at least 75%, or at least 80%, or at least 85%, or at least 90%, or at least 95%, or at least 97%, by weight of the composition. The liquid medium may comprise water, a non-aqueous solvent, or a mixture thereof, preferably water.

The composition 400 may comprise water. The composition may comprise at least 25%, or at least 50%, or at least 60%, or at least 70%, or at least 75%, or at least 80%, or at least 85%, or at least 90%, or at least 95%, or at least 97%, of water, by weight of the composition.

The composition 400 may comprise a non-aqueous solvent, preferably an organic solvent. Preferred organic solvents include monohydric alcohols, dihydric alcohols, polyhydric alcohols, glycerol, glycols including polyalkylene glycols such as polyethylene glycol, and mixtures thereof. More preferred non-aqueous solvents include monohydric alcohols, dihydric alcohols, polyhydric alcohols, glycerol, and mixtures thereof. Highly preferred are mixtures of solvents, especially mixtures of two or more of the following: lower aliphatic alcohols such as ethanol, propanol, butanol, isopropanol; diols such as 1,2-propanediol or 1,3-propanediol; and glycerol. Also preferred are propanediol and mixtures thereof with diethylene glycol, where the mixture contains no methanol or ethanol. Other suitable non-aqueous solvents may include alkanolamines, such as monoethanolamine or triethanolamine.

The compositions 400 further comprise a consumer product adjunct ingredient (“adjunct ingredient” or even “adjunct,” as used herein). The consumer product adjunct ingredients are typically benefit agents that provide a performance benefit in the end-use of the composition. Suitable consumer product adjunct ingredients may also include processing or stability aids.

The adjunct ingredient is generally insoluble in the liquid medium. For example, the adjunct ingredient may have a solubility of less than 10 g, or less than 5 g, or less than 1 g, or less than 0.5 g, or less than 0.1 g, per 1 L of liquid medium, at 20° C.

The adjunct ingredient may be in the form of particles, fibers, droplets, or a mixture thereof. The particles may be core/shell encapsulates, where a core that comprises a benefit agent is surrounded by a shell. The adjunct ingredient may characterized by a mean particle size of from about 1 micron to about 2 mm, preferably from about 5 micron to about 1 mm, more preferably from about 10 micron to about 100 micron.

The adjunct ingredient may comprise particles, for example, core/shell encapsulates, that are frangible. It is believed that the rotation cycles described herein are selected so as to minimize particle breakage or rupture during the re-homogenization processes. Rotation cycles that are characterized, for example, by higher rpms or more abrupt direction changes may adversely affect adjunct ingredients in the form of a frangible particle.

The composition 400 may comprise from about 0.5% to about 50%, or from about 1% to about 40%, or from about 1% to about 30%, or from about 1% to about 25%, or from about 1% to about 20%, or from about 1% to about 15%, or from about 1% to about 10%, or from about 1% to about 5%, by weight of the composition, of the adjunct ingredient.

The adjunct ingredient may be characterized by a density that is different from the density of the liquid medium, for example, a density that is more than or less than the density of the liquid medium. In such cases, the adjunct ingredient may tend to sink or float, respectively, in the liquid medium over time, resulting in inhomogeneity such as concentration differences at the top and bottom of the container. In such cases, mixing processes according to the present disclosure are likely to be particularly useful.

The adjunct ingredient may be a benefit agent that provides a desired benefit in consumer product. The adjunct ingredient may be selected from the group consisting of perfume delivery systems (preferably core/shell encapsulates), silicones, polymers, opacifiers, pearlescent agents, structurants or rheology modifier, particulate dyes and/or pigments, and mixtures thereof.

The adjunct ingredient may comprise a perfume delivery system. The perfume delivery system may comprise particles, such as core/shell encapsulates, where a shell surrounds a core. The core may include perfume raw materials and, optionally, a partitioning modifier such as isopropyl myristate. The shell may include a polymeric material, such as a material selected from melamine-based materials (e.g., melamine-formaldehyde), polyacrylamide, polyurea, polyurethanes, polyacrylate based materials, polyacrylate esters based materials, malic anhydride, polyamides, aromatic alcohols, polyvinyl alcohol, and mixtures thereof. The shell may include a coating on an outer surface, such as a coating comprising a cationic polymer, which may be useful to aid deposition.

The adjunct ingredient may comprise silicone. Useful silicones can be any suitable silicone-comprising compound. The silicone polymer may be selected from the group consisting of cyclic silicones, polydimethylsiloxanes, aminosilicones, cationic silicones, silicone polyethers, silicone resins, silicone urethanes, and mixtures thereof, preferably polydimethylsiloxanes, aminosilicones, or a mixture thereof. The silicone may comprise a polydialkylsilicone, such as a polydimethyl silicone (polydimethyl siloxane or “PDMS”), or a derivative thereof. The silicone may comprise an aminofunctional silicone, amino-polyether silicone, alkyoxylated silicone, cationic silicone, ethoxylated silicone, propoxylated silicone, ethoxylated/propoxylated silicone, quaternary silicone, or combinations thereof. The silicone may comprise a polydimethyl silicone, an aminosilicone, or a combination thereof, preferably an aminosilicone.

The adjunct ingredient may comprise a polymer. The polymer may be useful as a cleaning or dispersing agent, as a deposition aid, as a conditioning agent, as an aesthetic agent, or as a mixture thereof. The polymer may preferably be selected from polymeric soil release agents, amphiphilic graft co-polymers, cellulosic polymers, synthetic cationic polymers, polysaccharides, glyceride copolymers, dye-transfer-inhibitor polymers, or mixtures thereof.

The polymer may comprise cleaning and/or dispersing polymers, which may provide cleaning and/or whiteness benefits. Suitable cleaning and/or dispersing polymers may include: polymeric soil release agents, which may be anionic or nonionic and/or may include a terephthalate moiety; amphiphilic graft co-polymers, such as those derived from a polyethylene glycol backbone and having at least one pendant moiety selected from polyvinyl acetate, polyvinyl alcohol, or mixtures thereof (such as Sokalan HP22); cellulosic polymers, such as carboxymethyl cellulose, methyl cellulose, methyl hydroxyethyl cellulose, methyl carboxymethyl cellulose, and mixtures thereof; or mixtures thereof.

The polymer may comprise a cationic polymer, for example cationic polysaccharides, cationic proteins, or synthetic cationic polymers. Synthetic cationic polymers may include any of the known “polyquaternium” polymers identified by the International Nomenclature for Cosmetic Ingredients, such as polyquat-5, polyquat-6, polyquat-10, or polyquat-22. Suitable synthetic cationic polymers may include poly(acrylamide-co-diallyldimethylammonium chloride), poly(diallyldimethylammonium chloride-co-acrylic acid), poly(methylacrylamide-co-dimethylaminoethyl acrylate) and its quaternized derivatives, poly(vinylformamide-co-diallyldimethylammonium chloride-co-acrylic acid), poly(vinylformamide-co-diallyldimethylammonium chloride), poly(vinylpyrrolidone-co-acrylamide-co-vinyl imidazole) and its quaternized derivatives, poly(vinylpyrrolidone-co-methacrylamide-co-vinyl imidazole) and its quaternized derivatives, and mixtures thereof.

The polymer may comprise a polysaccharide, for example starch, cellulose, guar, chitosan, or a polyglucan. The polysaccharide may be a cationic polysaccharide. Cationic polysaccharides may include cationic cellulose derivatives, cationic guar gum derivatives, chitosan and derivatives, cationic starches, and/or cationic polyglucans. Suitable cationic polysaccharides include cationically modified cellulose, particularly cationic hydroxyethylcellulose and cationic hydroxypropylcellulose.

The polymer may comprise glyceride copolymers. The glyceride copolymers may be derived from natural oils. Examples of natural oils include, but are not limited to, vegetable oils, algae oils, fish oils, animal fats, tall oils, derivatives of these oils, combinations of any of these oils, and the like. The polymer may comprise polyhydroxystearic acid.

The polymer may comprise a dye transfer inhibitor (DTI) polymer. Suitable DTI polymers may include polyvinyl pyrrolidine (“PVP”), poly(4-vinylpyridine N-oxide) (“PVNO”), copolymers of PVP and PVNO, poly(N-vinyl-2-pyrrolidone)-poly(N-vinyl-imidazol) (“PVPVI”), or combinations thereof.

The adjunct ingredient may comprise opacifiers. Non-limiting examples of opacifiers may include: styrene/acrylate latexes; titanium dioxide (TiO₂); tin dioxide; any suitable form modified TiO₂, for example a coated TiO₂; or mixtures thereof.

The adjunct ingredient may comprise pearlescent agents. Non-limiting examples of pearlescent agents may include: mica; metal-oxide-coated mica; bismuth oxychloride; fish scales; mono and diesters of alkylene glycols; or mixtures thereof.

The adjunct ingredient may be sensitive to oxygen. For example, the adjunct ingredient may tend to auto-oxidize and decompose upon exposure to atmospheric air, which may lead to negatively perceived changes in color and/or odor. When the adjunct ingredient is sensitive to oxygen (e.g., by having a tendency to decompose via auto-oxidation), the processes, apparatuses, and systems of the present disclosure may be particularly useful because the container does not need to be opened and exposed to the atmosphere in order to mix the composition therein.

The composition may further comprise a processing aid, such as a solvent, structurant, dispersant, or a surfactant. The surfactant may include an anionic surfactant, a nonionic surfactant, or mixtures thereof. The composition may comprise neat perfume.

### Process of Mixing

The present disclosure relates to a process of mixing a composition in a closed or sealed container. The process generally relates to rotating the container in a horizontal plane around a vertical axis, typically with little to no vertical motion (e.g., little to no axial motion along the vertical axis). The process includes rotating the container in multiple rotation cycles, where a cycle includes rotating the container in a first direction (e.g., clockwise) and then in a second direction that is counter or opposite to the first direction (e.g., counter-clockwise). It is believed that by selecting rotation profiles with particular RPMs and rotation times, vortexes are created and collapsed in the liquid composition, leading to adequate mixing and homogenization.

The process may include providing a container 300 as described in more detail above. The container 300 may be sealed or otherwise closed. The container 300 may house a composition 400, where the composition 400 includes a liquid medium and an adjunct ingredient that is insoluble in the liquid medium. The container 300 may be relatively large, and may have an interior volume of at least 100 L. The container 300 may have a mass of at least 100 kilograms, inclusive of the composition 400 therein.

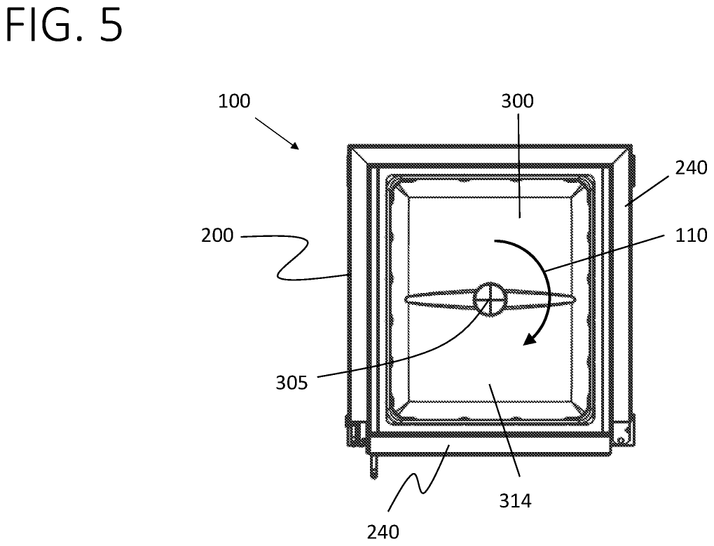

The process may include providing the container 300 to a mixing apparatus 200. Together, the mixing apparatus 200 and the container 300 may be part of a mixing system 100. As shown in FIGS. 4 and 5, the container 300 may be surrounded by a cage 240.

The process may include performing a rotation step that comprises at least two rotation cycles, where a rotation cycle includes rotating the container 300 (a) in a first direction 110 and then (b) in a second direction 120.

The rotation step may comprise at least three, preferably at least four, more preferably at least five rotation cycles. The rotation step may comprise from about two to about twenty rotation cycles, preferably from about three to about fifteen, more preferably from about five to about twelve, more preferably from about eight to eleven, most preferably about ten. It is believed that more rotation cycles will lead to improved mixing; however, too many can result in lost time and redundancy.

In total, the rotation step may last from about 1 minute to about 30 minutes, or from about 1 minute to about 20 minutes, or from about 1 minute to about 15 minutes, or from about 1 minute to about 10 minutes, preferably from about 2 minutes to about 8 minutes, more preferably from about 3 minutes to about 6 minutes.

A rotation cycle may comprise a first step that includes rotating the container 300 in a first direction 110. FIG. 5 shows a top view of the container 300 already having been provided to the cage 240 of a mixing apparatus 200. The container 300 may be rotated in a first direction 110, typically in a horizontal direction around a vertical axis 305 that is typically located at the center of the container 300, and typically is substantially orthogonal to the floor or ground.

The container 300 may be rotated in the first direction 110 for from about 5 to about 60 seconds, preferably from about 10 to about 45 seconds, more preferably from about 20 to about 40 seconds, even more preferably about 30 seconds.

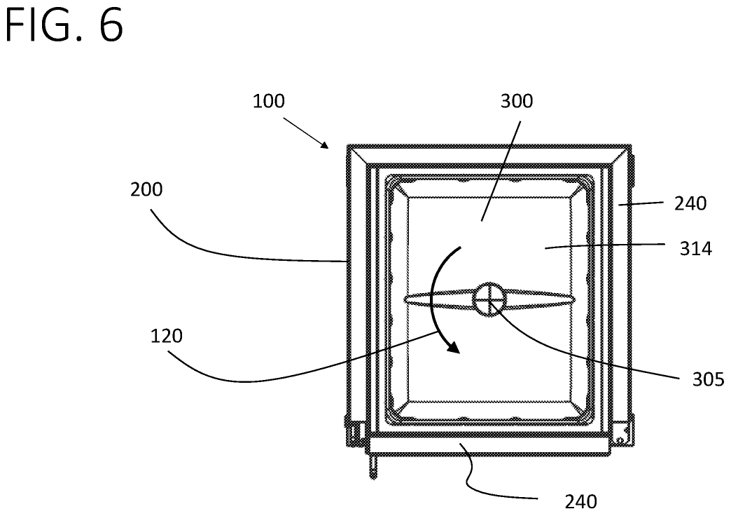

The rotation cycle may comprise a second step that includes rotating the container 300 in a second direction 120 that is counter or opposite to the first direction. FIG. 6 shows a top view of the container 300 already having been provided to the cage 240 of a mixing apparatus 200. The container 300 may be rotated in a second direction 120, typically around the vertical axis 305 of the container 300.

The container 300 may be rotated in the second direction for from about 5 to about 60 seconds, preferably from about 10 to about 45 seconds, more preferably from about 20 to about 40 seconds, even more preferably about 30 seconds.

The process may comprise rotating the containers 300 at a maximum speed, typically measured in rpms (revolutions per minute). The maximum speed of the first step, the second step, or both may be from about 20 rpms to about 100 rpms, preferably from about 30 rpms to about 80 rpms, more preferably from about 40 rpms to about 60 rpms, even more preferably about 50 rpms.

The first step of a rotation cycle, the second step, or both may comprise a ramp-up period, where the number of rpms is increasing; the ramp-up period may last from about 1 to about 15 seconds, preferably from about 3 to about 10 seconds, more preferably about 5 seconds.

The first step, the second step, or both may comprise a “hold” period, where the number of rpms stays constant, typically at the maximum rpms for the step or cycle; the hold period may last from about 1 to about 15 seconds, preferably from about 3 to about 10 seconds, more preferably about 5 seconds.

The first step, the second step, or both may comprise a ramp-down period, where the number of rpms decreases; the ramp-down period may last from about 1 to about 15 seconds, preferably from about 3 to about 10 seconds, more preferably about 5 seconds.

A rotation cycle may comprise a pause between the first step and the second step (e.g., between rotating in the first direction and the second direction). The pause may last from about 0.5 seconds to about 10 seconds, or from about 1 second to about 5 seconds, preferably about 1 second. It is preferred that the duration of the pause is relatively short, so that the composition does not have enough time to come to rest; it is believed that the turbulence resulting from the change in direction facilitates homogenization, and thus, the shorter the pause, the greater the turbulence and the better the mixing.

The rotation step may comprise a pause between rotation cycles, for example between a first rotation cycle and a second rotation cycle, or between a second and a third, etc. The pause may last from about 0.5 seconds to about 10 seconds, or from about 1 second to about 5 seconds, preferably about 1 second. It is preferred that the duration of the pause is relatively short, so that the composition does not have enough time to come to rest; it is believed that the turbulence resulting from the change in direction facilitates homogenization, and thus, the shorter the pause, the greater the turbulence and the better the mixing.

The process may result in an aeration degree of the composition of less than about 5%, or less than about 3%. Aeration is determined by comparing the regular/expected density of the composition (e.g., when made fresh) to the density following the rehomogenization process described herein. If the density is relatively lower upon rehomogenization, this is an indication that aeration has occurred. Lower degrees of aeration are typically desired for product stability and/or dosing reasons.

The composition 400 in the container 300 may be characterized by a viscosity difference between a sample taken from the top portion of the container (“top sample”) and the bottom portion of the container (“bottom sample”). After a rotation cycle, a rotation step, or the entire process, the difference in viscosity between a top sample and a bottom sample may be less than about 10%, or less than about 8%, or less than about 6%, or less than about 5%, or less than about 3%, or less than about 2%, where the larger viscosity is used as the denominator. Lower differences are preferred, as it indicates relatively better homogenization throughout the container.

The process of the present disclosure may be particularly suited to mixing a plurality of compositions in a plurality of containers. This is because the containers do not need to be opened or the compositions recirculated, so there is no clean out required of the equipment.

In the process of the present disclosure, the container 300 described above may be a first container, and the composition 400 may be a first composition. The process may further comprise providing a second container that houses a second composition and performing a rotation step on the second container, where the rotation step comprises two or more rotation cycles. The second composition may have a formulation that is the same as, or is different from, the first composition. The rotation cycles performed on the second container may be the same or different (e.g., with regard to duration and/or maximum rpms) as the rotation cycles of the rotation step performed on the first container.

The second composition may include a second liquid medium and a second adjunct ingredient that is insoluble in the second liquid medium. The second adjunct ingredient may be different from the adjunct ingredient (e.g., a first adjunct ingredient) that is present in the first composition. The concentration of the second adjunct ingredient in the second composition may be different from the concentration of the first adjunct ingredient in the first composition.

### Process of Making a Consumer Product

The present disclosure also relates to a process of making a consumer product. The consumer product may be useful for treating a surface, for example a fabric, a hard surface, hair, and/or skin.

The consumer product may in the form of a liquid composition, a granular composition, a hydrocolloid, a single-compartment pouch, a multi-compartment pouch, a dissolvable sheet, a pastille or bead, a fibrous article, a tablet, a stick, a bar, a flake, a foam/mousse, a non-woven sheet, or a mixture thereof.

The consumer product compositions of the present disclosure may be fabric care compositions, hard surface cleaner compositions, dish care compositions, hair care compositions, body cleansing compositions, or mixtures thereof. The compositions of the present disclosure may be fabric care compositions. Such compositions may be used as a pre-laundering treatment, a post-laundering treatment, or may be added during the rinse or wash cycle of the laundering operation. The consumer product composition may be selected from the group of heavy duty liquid detergent compositions, light duty liquid detergents compositions, detergent gels commonly used for laundry, bleaching compositions, laundry additives, fabric enhancer compositions, and mixtures thereof. The composition may be a compact heavy duty liquid detergent composition or a liquid fabric enhancer composition. Other non-limiting examples of liquid compositions according to the present disclosure include shampoos, body cleansing compositions, and the like.

The consumer product may be in the form of a unitized dose article, such as a pouch. Such pouches typically include a water-soluble film, such as a polyvinyl alcohol water-soluble film, that at least partially encapsulates a consumer product composition. Suitable films are available from MonoSol, LLC (Indiana, USA). The consumer product composition can be encapsulated in a single or multi-compartment pouch. A multi-compartment pouch may have at least two, at least three, or at least four compartments. A multi-compartmented pouch may include compartments that are side-by-side and/or superposed. The consumer product composition contained in the pouch or compartments thereof may be liquid, solid (such as powders), or combinations thereof; in such cases, at least one encapsulated consumer product composition is a liquid composition.

The process may include providing a composition that has been mixed according to the processes of the present disclosure to a base composition to make a consumer product.

The process of making a consumer product may comprise the step of mixing a composition housed in a container according to the processes of the present disclosure, and then combining a portion of the composition to a base composition, resulting in a consumer product.

The combining step may occur in a batch process, in a circulation loop process, and/or by an in-line mixing process, or even in a bottle via an in-situ mixing process. Suitable equipment for use in the processes disclosed herein may include continuous stirred tank reactors, homogenizers, turbine agitators, recirculating pumps, paddle mixers, plough shear mixers, ribbon blenders, vertical axis granulators and drum mixers, both in batch and, where available, in continuous process configurations, spray dryers, and extruders.

### Combinations

Specifically contemplated combinations of the disclosure are herein described in the following lettered paragraphs. These combinations are intended to be illustrative in nature and are not intended to be limiting.

A. A process for mixing a composition housed in a sealed container, the process comprising the steps of: providing a composition in a sealed container, wherein the composition comprises a liquid medium and a consumer product adjunct ingredient that is insoluble in the liquid medium; wherein the composition is present in an amount of (a) about 50 kg to about 5000 kg, and/or (b) an amount of about 50 L to about 5000 L; rotating the sealed container around a vertical axis in a rotating step, wherein the rotating step comprises at least two rotation cycles, wherein a rotation cycle comprises a first step followed by a second step, wherein the first step comprises rotating the container in a first direction around the vertical axis, for from about 5 to about 60 seconds at a maximum speed of from about 20 rpms to about 100 rpms, wherein the second step comprises rotating the container in a second direction around the vertical axis, for from about 5 to about 60 seconds at a maximum speed of from about 20 rpms to about 100 rpms, wherein the second direction is counter to the first direction.

B. The process according to paragraph A, wherein the rotation step comprises from about two to about twenty rotation cycles, preferably from about three to about fifteen, more preferably from about five to about twelve, more preferably from about eight to eleven, most preferably about ten.

C. The process according to either of paragraphs A or B, wherein the rotation step lasts from about 1 minute to about 30 minutes, or from about 1 minute to about 20 minutes, or from about 1 minute to about 15 minutes, or from about 1 minute to about 10 minutes, preferably from about 2 minutes to about 8 minutes, more preferably from about 3 minutes to about 6 minutes.

D. The process according to any of paragraphs A-C, wherein the maximum speed of the first step, the second step, or both is from about 30 rpms to about 80 rpms, more preferably from about 40 rpms to about 60 rpms, even more preferably about 50 rpms.

E. The process according to any of paragraphs A-D, wherein at least one of the following is true: (a) the first step comprises rotating the container in the first direction around the vertical axis, for from about 10 to about 45 seconds, more preferably from about 20 to about 40 seconds, even more preferably about 30 seconds; and/or (b) the second step comprises rotating the container in the second direction around the vertical axis, for from about 10 to about 45 seconds, more preferably from about 20 to about 40 seconds, even more preferably about 30 seconds.

F. The process according to any of paragraphs A-E, wherein the first step, the second step, or both, independently comprise one or more of the following: (a) a ramp-up period, where the number of rpms is increasing, preferably wherein the ramp-up period lasts from about 1 to about 15 seconds, preferably from about 3 to about 10 seconds, more preferably about 5 seconds; (b) a “hold” period, where the number of rpms stays constant, preferably wherein the hold period lasts from about 1 to about 15 seconds, preferably from about 3 to about 10 seconds, more preferably about 5 seconds; (c) a ramp-down period, where the number of rpms decreases, preferably wherein the ramp-down period lasts from about 1 to about 15 seconds, preferably from about 3 to about 10 seconds, more preferably about 5 seconds.

G. The process according to any of paragraphs A-F, wherein the rotation cycle comprises a pause between the first step and the second step, preferably wherein the pause between the first step and the second step lasts from about 0.5 seconds to about 10 seconds, or from about 1 second to about 5 seconds, preferably about 1 second.

H. The process according to any of paragraphs A-G, wherein the rotation step comprises a pause between rotation cycles, preferably wherein the pause between rotation cycles lasts from about 0.5 seconds to about 10 seconds, or from about 1 second to about 5 seconds, preferably about 1 second.

I. The process according to any of paragraphs A-H, wherein the composition is present in the amount of: (a) from about 100 kg to about 4000 kg, or from about 250 kg to about 3000 kg, or from about 500 kg to about 2000 kg, or from about 750 kg to about 1500 kg, or about 800 kg to about 1200 kg; and/or (b) from about 100 L to about 4000 L, or from about 250 L to about 3000 L, or from about 500 L to about 2000 L, or from about 750 L to about 1500 L, or about 800 L to about 1200 L.

J. The process according to any of paragraphs A-I, wherein the liquid medium comprises water, preferably wherein water is present at a level of at least 25%, or at least 50%, or at least 60%, or at least 70%, or at least 75%, or at least 80%, or at least 85%, or at least 90%, or at least 95%, or at least 97%, of water, by weight of the composition.

K. The process according to any of paragraphs A-J, wherein the composition comprises from about 0.5% to about 50%, or from about 1% to about 40%, or from about 1% to about 30%, or from about 1% to about 25%, or from about 1% to about 20%, or from about 1% to about 15%, or from about 1% to about 10%, or from about 1% to about 5%, by weight of the composition, of the consumer product adjunct ingredient.

L. The process according to any of paragraphs A-K, wherein the consumer product adjunct ingredient is selected from the group consisting of a perfume delivery system, silicones, polymers, opacifiers, pearlescent agents, and mixtures thereof.

M. The process according to any of paragraphs A-L, wherein the consumer product adjunct comprises a perfume delivery system, preferably core/shell encapsulates.

N. The process according to any of paragraphs A-M, wherein the consumer product adjunct comprises a polymer, preferably a cationic polymer, more preferably a cationic polysaccharide.

O. The process according to any of paragraphs A-N, wherein the consumer product adjunct ingredient is in the form of particles, fibers, droplets, or a mixture thereof.

P. The process according to any of paragraphs A-O, wherein the adjunct ingredient is characterized by a mean particle size of from about 1 micron to about 2 mm, preferably from about 5 microns to about 1 mm, more preferably from about 10 micron to about 100 micron.

Q. The process according to any of paragraphs A-P, wherein the composition, when homogenized, is characterized by a viscosity of from about 100 to about 10,000 cps, or from about 200 to about 8,000 cps, or from about 300 to about 5000 cps, or from about 500 to about 4000 cps.

R. The process according to any of paragraphs A-Q, wherein the sealed container is characterized by one or more of the following: (a) a footprint that is substantially rectangular; and/or (b) a shape that is substantially a rectangular prism.

S. The process according to any of paragraphs A-R, wherein the sealed container is provided to a mixing apparatus, wherein the mixing apparatus comprises a frame, wherein the frame supports a deck that is capable of rotating in the first direction and the second direction, relative to the frame, wherein the sealed container rests upon the deck, preferably wherein the deck further comprises one or more support structures to hold the container in place during a rotation cycle.

T. The process according to any of paragraphs A-S, wherein the sealed container is a first sealed container, and the composition is a first composition, wherein the process further comprises the step of providing a second sealed container that houses a second composition and rotating the second sealed container around a vertical axis in a second rotating step, wherein the second rotating step comprises at least two rotation cycles, each rotation cycle comprising a first step followed by a second step as described above, wherein the second composition comprises a second liquid medium and a second consumer product adjunct that is insoluble in the second liquid medium.

U. The process according to paragraph T, wherein the second consumer product adjunct is different from the consumer product adjunct of the first composition.

V. The process according to any of paragraphs A-U, wherein the container further comprises a headspace present at a level of from about 5% to about 20%, preferably from about 10% to about 20%, even more preferably from about 12% to about 18%, by volume, of the internal volume 315 of the container.

W. The process according any of paragraphs A-V, wherein after the process of mixing is completed, the composition is characterized by a difference in viscosity between a top sample and a bottom sample that is less than about 10%, or less than about 8%, or less than about 6%, or less than about 5%, or less than about 3%, or less than about 2%, where the larger viscosity is used as the denominator.

X. A process of making a consumer product, wherein the process comprises the steps of: mixing a composition by a process according to any of paragraphs A-W; and combining the composition with a base composition to make the consumer product.

### Test Methods

### Viscosity

To determine viscosity of a liquid composition, a Brookfield rotational viscometer (model DV-1) is used. A sample (approx. 100 mL) of the composition is poured into a plastic container, and the viscometer spindle is placed inside the container until the spindle is submerged up to the mark. At a rotation rate of 60 rpms, spindle LV-3 is used for compositions that have a viscosity of up to 2000 cps; if the composition has a viscosity greater than 2000 cps, the spindle LV-4 is used. The machine is turned on and is allowed to spin until the viscosity value stabilizes, typically after two minutes. The viscosity value is recorded. The measurements are taken at approx. 22° C.

### Collecting Top and Bottom Samples

Samples of the liquid compositions may be taken from the top and bottom of the container.

To collect a sample from the top of the container, the lid is removed, and a cup (volume of about 200 mL) is immersed about 10 cm below the surface of the liquid and then removed. A portion of the sample may be transferred to a different container for analysis.

To collect a sample from the bottom of the container, the valve is first purged by opening the valve and allowing approximately 500 g of material to flow out of the container. After purging, a sample of approximately 500 g of material is collected in a container. A portion of the sample may be transferred to a different container for analysis.

After collection, the samples may be tested for viscosity, density, or any other suitable characteristic.

### Composition Density

The method for determining the density of the liquid composition is based on ASTM D1475-13.

A clean and dry density cup (pycnometer—100 mL) is placed on a scale; the scale is tared/zeroed. The composition sample is added to the cup carefully so as not to create air bubbles; the cup is filled to the top. The cup lid is put into place, and excess liquid is removed from the top hole. The full cup is weighed, and the value is recorded. The measurements are taken at approx. 22° C.

### Particle Size

Particle size is preferably reported as the volume-weighted median particle size. If not available, then number-weighted median particle size may be reported.

The method used for determining particle size may vary on the type of materials and size of particles being analyzed. One of ordinary skill may determine the most appropriate method under the circumstances, but for the following materials, the methods provided below are used.

A. Perfume Encapsulates (5-100 Microns)

The size of perfume encapsulates is determined via single-particle optical sensing (SPOS), also called optical particle counting (OPC), using the AccuSizer 780 AD instrument and the accompanying software CW788 version 1.82 (Particle Sizing Systems, Santa Barbara, Calif., U.S.A.), or equivalent. The instrument is configured with the following conditions and selections: Flow Rate=1 ml/sec; Lower Size Threshold=0.50 μm; Sensor Model Number=LE400-05 or equivalent; Autodilution=On; Collection time=60 sec; Number channels=512; Vessel fluid volume=50 ml; Max coincidence=9200. The measurement is initiated by putting the sensor into a cold state by flushing with water until background counts are less than 100. A sample of delivery capsules in suspension is introduced, and its density of capsules adjusted with DI water as necessary via autodilution to result in capsule counts of at least 9200 per ml. During a time period of 60 seconds the suspension is analyzed. The range of size used was from 1 um to 493.3 μm. Accordingly, the volume distributions and number distributions are calculated as shown and described above.

From the cumulative volume distribution, also the diameter of the percentiles 5 (d₅), 50 (d₅₀) and 90 (d₉₀) can be calculated (Percentile j is determined by the cumulative volume distribution where the j percentage of the volume is accumulated (Σ_{d=1 um}^{d}^{ⱼ}x_{i,ν}=j (%)).

B. Silicone Droplets

The droplet size for the silicone compounds are analyzed utilizing a Horiba, Partica, Laser Scattering, Particle Size Distribution Analyzer LA-950V2 with a static quartz cell and operated in accordance with the manufacturer's instructions.

C. Cationic Cellulose Particles

The Occhio Flow Cell FC200-S (Angleur, Belgium) is used to measure the particle size distribution. The sample containing the particles to be analysed is diluted to 2% by weight, using PEG200, to ensure single particle detection. 2 ml of the diluted sample is analysed according to the instructions provided with the device.

### EXAMPLES

The examples provided below are intended to be illustrative in nature and are not intended to be limiting.

### Example 1. Illustrative Mixing Process

A container 300 as shown in FIG. 3 is provided to a mixing apparatus 200 to make a system 100 as shown in FIGS. 1 and 2. The container 300 includes a composition in the form of an aqueous slurry, including water as a liquid medium. The composition further includes perfume encapsulates, where the core-shell encapsulates are characterized by a volume weighted particle size of approximately 18 microns. The walls of the encapsulates include polyacrylate polymer, and perfume raw materials (as benefit agent) and isopropyl myristate (as partitioning modifier) are found in the core. The encapsulates are present at a level of about 35% by weight of the composition, and about 1000 kg of the composition is present in the container 300. The dimensions of the container 300 are approximately 1.2 m (length)×1.0 m (width)×1.15 m (height). Additional materials, such as structurant and/or preservatives, may be present in the composition.

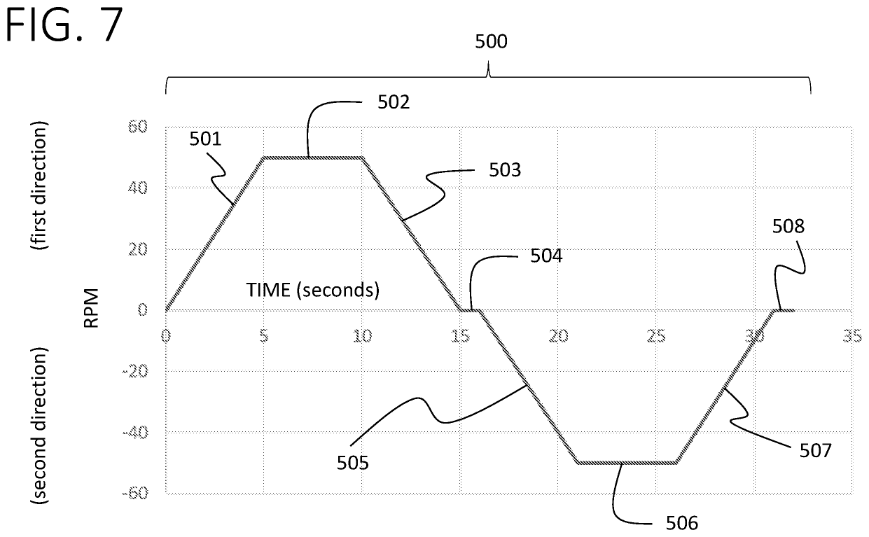

Once the container 300 is safely loaded onto the mixing apparatus 200, the apparatus 200 rotates the container 300 in a series of rotation cycles. FIG. 7 shows a graph of a typical rotation cycle 500. Time (in seconds) is shown on the x-axis, and RPMs is shown on the y-axis, with revolutions in the first direction shown as positive RPMs and revolutions in the second direction shown as negative RPMs. The entire rotation cycle lasts thirty-two seconds.

As shown in FIG. 7, the first step of the rotation cycle 500 lasts fifteen seconds (from time 0 to time 15). The first step includes a ramp-up period 501 of five seconds in the first direction, a hold period 502 of five seconds with a maximum RPM of 50 RPMs, and a ramp-down period 503 of five seconds. The rotation cycle includes a pause 504 of one second between the first step and the second step.

The second step of the rotation cycle 500 also lasts fifteen seconds (from time 16 to time 31). The second step of the rotation cycle 500 shows a ramp-up period 505 of five seconds in the second direction, a hold period 506 of five seconds with a maximum RPM of −50 RPMs (i.e., 50 RPMs in the second direction), and a ramp-down period 507 of five seconds. The rotation cycle includes a pause 508 of one second at the end of the rotation cycle 500, before the next rotation cycle begins.

The process includes ten of these rotation cycles 500 in series, so that the rotation cycles last a total of approximately 320 seconds. The container 300 is removed from the apparatus 200, and shortly thereafter a portion of the perfume encapsulate composition contained therein is added to a liquid fabric enhancer base composition via in-line mixing. The process results in a consumer product, specifically in a liquid fabric enhancer product.

### Example 2. Viscosity Differences Before and After Mixing

For this example, container systems comprising perfume microcapsule feedstock compositions are provided. The perfume microcapsules (PMCs) comprise melamine-formaldehyde walls, and the walls are coated with polyvinyl formamide as a deposition aid. The microcapsules labeled PMC 1 contain a first mixture of perfume raw materials, and the microcapsules labeled PMC 2 contain a second, different mixture of perfume raw materials. The tested PMC compositions comprise about 45% wt % of particles (i.e., the perfume microcapsules).

The PMC compositions are manufactured at a first location, provided to a rectangular container as described in Example 1, and transported to a second location, where the viscosity testing and mixing/re-homogenization processes occur.

Viscosities, measured in cps on a Brookfield viscometer, of the composition found at the top of the container and at the bottom are assessed at various times, including prior to re-homogenization/mixing (“Initial”), and after 10, 20, 30, and 40 rotation cycles of mixing according to the present disclosure, where a rotation cycle is as provided in Example 1 and FIG. 7. The difference in top and bottom viscosity are also determined, expressed as a percentage difference using the larger viscosity value as the denominator. The results are provided in Table 1 below.

TABLE 1

| | | Viscosity (in cps) | | | | |
|---|---|---|---|---|---|---|
| | | | After 10 | After 20 | After 30 | After 40 |
| | | Initial | cycles | cycles | cycles | cycles |
| PMC 1 | Top | 2999 | 3199 | 3195 | 3015 | 3235 |
| | Bottom | 3575 | 3259 | 3259 | 3179 | 3519 |
| | Viscosity | 16.11% | 1.84% | 1.96% | 5.16% | 8.07% |
| | difference | | | | | |
| | top/bottom | | | | | |
| PMC 2 | Top | 2895 | 1400 | 1336 | 1232 | 826 |
| | Bottom | 4 | 1020 | 1356 | 1400 | 882 |
| | Viscosity | 99.86% | 27.14% | 1.47% | 12.00% | 6.35% |
| | difference | | | | | |
| | top/bottom | | | | | |

As shown in Table 1 above, prior to re-homogenization, the compositions (after manufacture and transport) are relatively heterogeneous in terms of viscosity at the top compared to the bottom of the container. The composition containing PMC 1 is found to have a lower viscosity at the top of the container compared to the bottom; the composition containing PMC 2 is found to have phase-split almost completely, where the PMCs are generally found in a layer near the top of the composition.

As shown in Table 1, the viscosity differences between the top and the bottom of each PMC composition are reduced after ten and twenty rotation cycles. Preferably, the viscosity difference is 10% or less, more preferably 5% or less.

As also shown in Table 1, the viscosity differences of the compositions relatively increased after thirty and forty cycles, compared to the viscosity difference after twenty cycles. This suggests that it may be preferred to control or minimize the number of rotation cycles.

Similar tests are performed on an expired batch of perfume microcapsules. However, the viscosity at the top of the container prior to re-homogenization was quite high and could not be measured with the viscometer at hand. Still, after forty rotation cycles according to Example 1, the viscosity difference between the top and bottom of the container is 10.40%.

Additional tests show that re-homogenization of a PMC slurry using the rotation cycle described above results in little-to-no increase of free perfume oil in the slurry. This indicates that PMCs are generally not rupturing during a re-homogenization process according to the present disclosure.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A process for mixing a composition housed in a sealed container, the process comprising the steps of:
providing a composition in a sealed container,
wherein the composition comprises a liquid medium and a consumer product adjunct ingredient that is insoluble in the liquid medium;
wherein the composition is present in an amount of
(a) about 50 kg to about 5000 kg, and/or
(b) an amount of about 50 L to about 5000 L;
rotating the sealed container around a vertical axis in a rotating step,
wherein the rotating step comprises at least two rotation cycles,
wherein a rotation cycle comprises a first step followed by a second step,
wherein the first step comprises rotating the container in a first direction around the vertical axis, for from about 5 to about 60 seconds at a maximum speed of from about 20 rpms to about 100 rpms,
wherein the second step comprises rotating the container in a second direction around the vertical axis, for from about 5 to about 60 seconds at a maximum speed of from about 20 rpms to about 100 rpms,
wherein the second direction is counter to the first direction.

2. The process according to claim 1, wherein the rotation step comprises from about two to about twenty rotation cycles.

3. The process according to claim 1, wherein the rotation step lasts from about 1 minute to about 30 minutes.

4. The process according to claim 1, wherein the maximum speed of the first step, the second step, or both is from about 30 rpms to about 80 rpms.

5. The process according to claim 1, wherein at least one of the following is true:
(a) the first step comprises rotating the container in the first direction around the vertical axis, for from about 10 to about 45 seconds; and/or
(b) the second step comprises rotating the container in the second direction around the vertical axis, for from about 10 to about 45 seconds.

6. The process according to claim 1, wherein the first step, the second step, or both, independently comprise one or more of the following:
(a) a ramp-up period, where the number of rpms is increasing, wherein the ramp-up period lasts from about 1 to about 15 seconds;
(b) a “hold” period, where the number of rpms stays constant, wherein the hold period lasts from about 1 to about 15 seconds;
(c) a ramp-down period, where the number of rpms decreases, wherein the ramp-down period lasts from about 1 to about 15 seconds.

7. The process according to claim 1, wherein the rotation cycle comprises a pause between the first step and the second step, wherein the pause between the first step and the second step lasts from about 0.5 seconds to about 10 seconds.

8. The process according to claim 1, wherein the rotation step comprises a pause between rotation cycles, wherein the pause between rotation cycles lasts from about 0.5 seconds to about 10 seconds.

9. The process according to claim 1, wherein the composition is present in the amount of:
(a) from about 100 kg to about 4000 kg; and/or
(b) from about 100 L to about 4000 L.

10. The process according to claim 1, wherein the container further comprises a headspace present at a level of from about 5% to about 20%, by volume, of the internal volume of the container.

11. The process according to claim 1, wherein the liquid medium comprises water, wherein water is present at a level of at least 25%, by weight of the composition.

12. The process according to claim 1, wherein the composition comprises from about 0.5% to about 50%, by weight of the composition, of the consumer product adjunct ingredient.

13. The process according to claim 1, wherein the consumer product adjunct ingredient is selected from the group consisting of a perfume delivery system, silicones, polymers, opacifiers, pearlescent agents, and mixtures thereof.

14. The process according to claim 1, wherein the consumer product adjunct comprises a perfume delivery system, wherein the perfume delivery system comprises core/shell encapsulates.

15. The process according to claim 1, wherein the consumer product adjunct comprises a polymer.

16. The process according to claim 1, wherein the consumer product adjunct ingredient is in the form of particles, fibers, droplets, or a mixture thereof.

17. The process according to claim 1, wherein the adjunct ingredient is characterized by a mean particle size of from about 1 micron to about 2 mm.

18. The process according to claim 1, wherein the composition, when homogenized, is characterized by a viscosity of from about 100 to about 10,000 cps.

19. The process according to claim 1, wherein after the process of mixing is completed, the composition is characterized by a difference in viscosity between a top sample and a bottom sample that is less than about 10%, where the larger viscosity is used as the denominator.

20. The process according to claim 1, wherein the sealed container is characterized by one or more of the following:
(a) a footprint that is substantially rectangular; and/or
(b) a shape that is substantially a rectangular prism.

21. The process according to claim 1, wherein the sealed container is provided to a mixing apparatus,
wherein the mixing apparatus comprises a frame,
wherein the frame supports a deck that is capable of rotating in the first direction and the second direction, relative to the frame, wherein the sealed container rests upon the deck.

22. The process according to claim 1,
wherein the sealed container is a first sealed container, and the composition is a first composition,
wherein the process further comprises the step of providing a second sealed container that houses a second composition and rotating the second sealed container around a vertical axis in a second rotating step,
wherein the second rotating step comprises at least two rotation cycles, each rotation cycle comprising a first step followed by a second step as described above,
wherein the second composition comprises a second liquid medium and a second consumer product adjunct that is insoluble in the second liquid medium.

23. The process according to claim 22, wherein the second consumer product adjunct is different from the consumer product adjunct of the first composition.

24. A process of making a consumer product, wherein the process comprises the steps of:
mixing a composition by the process according to claim 1;
combining the composition with a base composition to make the consumer product.
